(19) United States
(12) Patent Application Publication
(10) Pub. No.: US 2021/0196652 A1
(43) Pub. Date: Jul. 1, 2021

(54) USE OF CYSTEAMINE AND DERIVATIVES THEREOF TO TREAT DYSFUNCTIONAL TEAR SYNDROME (DTS)

(71) Applicant: MESHABERASE, LLC, Potomac, MD (US)

(72) Inventor: Benjamin RUBIN, Potomac, MD (US)

(73) Assignee: MESHABERASE, LLC, Potomac, MD (US)

(21) Appl. No.: 17/119,000

(22) Filed: Dec. 11, 2020

Related U.S. Application Data
(60) Provisional application No. 62/954,156, filed on Dec. 27, 2019.

(51) Int. Cl. A61K 31/131; A61K 45/06; A61P 27/04; A61K 47/10; A61K 9/00; A61K 9/08

(52) U.S. Cl. CPC A61K 31/131; A61K 45/06; A61K 9/08; A61K 47/10; A61K 9/0048; A61P 27/04

(57) ABSTRACT

Methods for treating or inhibiting dysfunctional tear syndrome (DTS) or symptoms associated therewith, and compositions for use in the methods.

This application claims priority to U.S. provisional application No. 62/954,156, filed Dec. 27, 2019, the entire disclosure of which is incorporated herein by reference.

### FIELD OF THE INVENTION

The present invention relates to the use of cysteamine or cystamine, or derivatives thereof, to treat, inhibit, or suppress dysfunctional tear syndrome (DTS).

### BACKGROUND

Dysfunctional tear syndrome is the condition of having dry eyes. It is one of the most common reasons for seeking medical care from an ophthalmologist, as it can cause chronic and debilitating eye pain, with the cornea being one of the most densely sensory-neuron-innervated parts of the body.

Dysfunctional tear syndrome can also significantly affect visual acuity. In this regard, DTS results in transient epithelial and tear film irregularities. As the cornea is highly specialized to refract and transmit light, even minute irregularities can degrade optical quality. Patients with dysfunctional tear syndrome report difficulty in reading newspapers and road signs, driving at night, navigating stairs, cooking, watching television, and even recognizing friends.

Given the critical role the human eye plays in daily life, dysfunctional tear syndrome is associated with general job and living limitations, as well as lower vitality and quality of life. Dysfunctional tear syndrome is far more than a nuisance condition—it is life changing, and its treatment is critical to improving the lives of millions of people.

### SUMMARY

In one aspect, described herein is a method for reducing at least one symptom of dysfunctional tear syndrome (DTS) or ocular lubrication dysfunction by methods including administering cysteamine, or a derivative thereof, to a subject exhibiting at least one symptom of DTS or ocular lubrication dysfunction. The subject may be a human, and in some embodiments may be a human diagnosed with recurrent corneal erosion or keratoconjunctivitis sicca.

In some embodiments, the method involves administering cysteamine at least once daily; in other embodiments, the method involves administering cysteamine at least twice daily. In some embodiments, the dosage may range from about 0.01 to about 2.0 mg/ml cysteamine.

In some embodiments, the administered composition will include cysteamine in saline. The composition may include cysteamine in combination with at least one additional active agent, such as, for example, an(other) MMP-9 inhibitor.

In some embodiments, the administered composition will include cysteamine and at least one pharmaceutically acceptable excipient for alleviating the symptoms of dysfunctional tear syndrome (DTS). The composition may include, for example, an excipient such as glycerol.

The foregoing summary is not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section.

In addition to the foregoing, the invention includes, as an additional aspect, all embodiments of the invention narrower in scope in any way than the variations defined by specific paragraphs above. For example, certain aspects of the invention that are described as a genus, and it should be understood that every member of a genus is, individually, an aspect of the invention. Also, aspects described as a genus or selecting a member of a genus, should be understood to embrace combinations of two or more members of the genus. Although the applicant(s) invented the full scope of the invention described herein, the applicants do not intend to patent subject matter described in the prior art work of others. Therefore, if statutory prior art within the scope of a claim is brought to the attention of the applicant(s) by a Patent Office or other entity or individual, the applicant(s) reserve the right to exercise amendment rights under applicable patent laws to redefine the subject matter of such a paragraph to specifically exclude such statutory prior art or obvious variations of statutory prior art from the scope of such a paragraph. Variations of the invention defined by such amended paragraphs also are intended as aspects of the invention.

### BRIEF DESCRIPTION OF THE FIGURES

FIG. 1 summarizes daily Likert scores for the right eyes of treated patients.

FIG. 2 summarizes daily Likert scores for the left eyes of treated patients.

### DETAILED DESCRIPTION

The present disclosure relates, in general, to the discovery of the effects of cysteamine in treatment of dysfunctional tear syndrome (DTS).

### Definitions

As used herein and in the appended claims, the singular forms “a,” “and” and “the” include plural referents unless the context dictates otherwise Thus, for example, reference to “a derivative” includes a plurality of such derivatives and reference to “a patient” includes reference to one or more patients and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be described using language “consisting essentially of” or “consisting of.”

Unless defined otherwise, all technical and scientific terms used have the same meaning as commonly understood to one of ordinary skill in the art to which this disclosure belongs. Although methods and materials similar or equivalent to those described herein can be used in the practice of the disclosed methods and products, the exemplary methods, devices and materials are described herein.

The documents discussed above and throughout the text are provided solely for their disclosure before the filing date of the present application. Nothing herein is to be construed as an admission that the inventors are not entitled to antedate such disclosure by virtue of prior disclosure. Each document is incorporated by reference in its entirety with particular attention to the disclosure for which it is cited.

The following references provide one of skill with a general definition of many of the terms used in this disclosure: Singleton, et al., DICTIONARY OF MICROBIOLOGY AND MOLECULAR BIOLOGY (2d ed 1994); THE CAMBRIDGE DICTIONARY OF SCIENCE AND TECHNOLOGY (Walker ed, 1988); THE GLOSSARY OF GENETICS, 5TH ED., R Rieger, et al. (eds.). Springer Verlag (1991), and Hale and Marham, THE HARPER COLLINS DICTIONARY OF BIOLOGY (1991).

“Dysfunctional tear syndrome” (DTS) is a condition characterized by one or more dysfunctions in tear production, including low volume tear production (with normal quality tears), or excessive tear production with poor quality tears (e.g., due to water content compared to low oil and mucous content), or any combination of these features. It is synonymous with ocular lubrication dysfunction. (Throughout the specification, reference to dysfunctional tear syndrome includes ocular lubrication dysfunction, and vice versa.) DTS includes in some embodiments keratitis sicca and recurrent conical erosion. In some cases, DTS will be related to an increased activity or concentration of matrix metalloproteinases (MMP's). In sonic instances, it can be accompanied by erosion of surface tissue. The terms recurrent corneal erosion, recurrent epithelial erosion, and recurrent corneal epithelial erosion may be used interchangeably in this application.

As used herein, a “therapeutically effective amount” or “effective amount” refers to that amount of a cysteamine product, e.g., cysteamine, cystamine, or a pharmaceutically acceptable salt thereof, sufficient to result in amelioration of symptoms, for example, treatment, healing, prevention or amelioration of the relevant medical condition, or an increase in rate of treatment, healing, prevention or amelioration of such conditions, typically providing a statistically significant improvement in the treated patient population. When referencing an individual active ingredient, administered alone, a therapeutically effective dose refers to that ingredient alone. When referring to a combination, a therapeutically effective dose refers to combined amounts of the active ingredients that result in the therapeutic effect, whether administered in combination, including serially or simultaneously. In various embodiments, a therapeutically effective amount of the cysteamine product ameliorates symptoms associated with dysfunctional tear syndrome (DTS).

“Treatment” refers to prophylactic treatment or therapeutic treatment. In certain embodiments, “treatment” refers to administration of a compound or composition to a subject for therapeutic or prophylactic purposes.

“Therapeutic” treatment is a treatment administered to a subject who exhibits signs or symptoms of pathology for the purpose of diminishing or eliminating those signs or symptoms. The signs or symptoms may be biochemical, cellular, histological, functional or physical, subjective or objective.

As used herein, “prevent” means to lessen the likelihood of some event. For example, to prevent dysfunctional tear syndrome (DTS) means to lessen the likelihood of DTS; to prevent a symptom of dysfunctional tear syndrome (DTS) means to lessen the likelihood of a symptom of DTS. “Prevent” does not require complete, or 100% inhibition or reduction, as that level of inhibition or reduction is simply unrealistic in biological environments. When the inventor intends to convey that 100% reduction of a symptom is intended, the inventor will expressly state that fact.

A “prophylactic” treatment is a treatment administered to a subject who does not exhibit signs of a disease or exhibits only early signs of the disease, for the purpose of decreasing the risk of developing pathology. The compounds or compositions of the disclosure may be given as a prophylactic treatment to reduce the likelihood of developing a pathology or to minimize the severity of the pathology, if developed. As with “prevent,” “prophylaxis” does not require 100% prevention to be considered effectively prophylactic.

“Diagnostic” means identifying the presence, extent and/or nature of a pathologic condition. Diagnostic methods differ in their specificity and selectivity. While a particular diagnostic method may not provide a definitive diagnosis of a condition, it suffices if the method provides a positive indication that aids in diagnosis.

“Pharmaceutical composition” refers to a composition suitable for pharmaceutical use in a subject animal, including humans and mammals. A pharmaceutical composition comprises a therapeutically effective amount of a cysteamine product, optionally another biologically active agent, and optionally a pharmaceutically acceptable excipient, carrier or diluent. In an embodiment, a pharmaceutical composition encompasses a composition comprising the active ingredient(s), and the inert ingredient(s) that make up the carrier, as well as any product that results, directly or indirectly, from combination, complexation or aggregation of any two or more of the ingredients, or from dissociation of one or more of the ingredients, or from other types of reactions or interactions of one or more of the ingredients. The pharmaceutical compositions of the present disclosure encompass any composition made by admixing a compound of the disclosure and a pharmaceutically acceptable excipient, carrier or diluent.

“Pharmaceutically acceptable carrier” refers to any of the standard pharmaceutical carriers, buffers, and the like, such as a phosphate buffered saline solution, 5% aqueous solution of dextrose, and emulsions (e.g., an oil/water or water/oil emulsion). Non-limiting examples of excipients include adjuvants, binders, fillers, diluents, disintegrants, emulsifying agents, wetting agents, lubricants, glidants, sweetening agents, flavoring agents, and coloring agents. Suitable pharmaceutical carriers, excipients and diluents are described in Remington's Pharmaceutical Sciences, 19th Ed. (Mack Publishing Co., Easton, 1995). Preferred pharmaceutical carriers depend upon the intended mode of administration of the active agent. Typical modes of administration include enteral (e.g., oral) or parenteral (e.g., subcutaneous, intramuscular, intravenous or intraperitoneal injection; or topical, transdermal, or transmucosal administration).

A “pharmaceutically acceptable salt” is a salt that can be formulated into a compound for pharmaceutical use, including but not limited to metal salts (e.g., sodium, potassium, magnesium, calcium, etc.) and salts of ammonia or organic amines. Examples of cysteamine derivatives include hydrochloride, bitartrate, and phosphocysteamine derivatives. Cystamine and cystamine derivatives include sulfated cystamine.

As used herein “pharmaceutically acceptable” or “pharmacologically acceptable” salt, ester or other derivative of an active agent comprise, for example, salts, esters or other derivatives refers to a material that is not biologically or otherwise undesirable, i.e., the material may be administered to an individual without causing any undesirable biological effects or without interacting in a deleterious manner with any of the components of the composition in which it is contained or with any components present on or in the body of the individual.

As used herein, the term “unit dosage form” refers to physically discrete units suitable as unitary dosages for human and animal subjects, each unit containing a predetermined quantity of a compound of the disclosure calculated in an amount sufficient to produce the desired effect, optionally in association with a pharmaceutically acceptable excipient, diluent, carrier or vehicle. The specifications for the novel unit dosage forms of the present disclosure depend on the particular compound employed and the effect to be achieved, and the pharmacodynamics associated with each compound in the host.

As used herein, the term “subject” encompasses mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats, laboratory animals including rodents, such as rats, mice and guinea pigs, and the like. The term does not denote a particular age or gender. In various embodiments the subject is human.

As a disease, dysfunctional tear syndrome (DTS) (or ocular lubrication dysfunction) is poorly understood. Its symptoms are easily identified, particularly in the patents themselves, with patients' assessments of the symptoms often exceeding physicians' assessments. Fluorescein, and other dyes, have been used by physicians to identify the presence of corneal tissue affected DTS, but the dyes are often simply confirmatory of the condition and symptoms already recognized by the patients. Treatments have focused on minimizing environmental causes, improving hydration, conserving tears, and other generally palliative efforts. But treatments that address the underlying cause of the disease have been lacking.

In 1969, Dohlman and his co-workers identified metalloproteinases as substances produced in ocular surface diseases that were important contributors to corneal ulceration, destruction, and perforation (Slansky H H, Gnädinger M C, Itoi M, et al “Collagenase in corneal ulcerations.” Arch Ophthalmol. 1969, 82:108-111; see also Berman M, Dohlman C H, Gnädinger M, et al. “Characterization of collagenolytic activity in the ulcerating cornea,” Exp Eye Res 1971; 11:255-257; and Pfister R R, McCulley J P, Friend J. et al. “Collagenase activity of intact corneal epithelium in peripheral alkali burns,” Arch Ophthalmol. 1971; 86: 308-313.) A number of matrix metalloproteinases (MMPs) have been identified since that time, and they are believed to be capable of dissolving collagen and other tissues. MMP-9 has been particularly implicated in ocular pathologies. (See, for example, U.S. Pat. No. 7,652,070.)

Numerous reports have documented the role of matrix metalloproteinases in ocular surface disease, as a marker of disease and as an agent of pathology. (Kauffman, “The Practical Detection of MMP-9 Diagnoses Ocular Surface Disease and May Help Prevent Its Complications,” Cornea Vol. 32, No. 2, February 2012: 211-216.) For the first time, a new in-office immunoassay for the detection of MMP-9 is available. (See Kaufman.) The assay can be of great value for both the detection of ocular surface disease and the prevention of complications after laser in situ keratomileusis (LASIK) and other ophthalmic surgeries, as the present invention provides a safe ocular MMP inactivator.

Cysteamine is an approved ocular medication for cystinosis and is approved as a treatment of the eve. However, when administered for conditions such as cystinosis, cysteamine has been reported to cause slinging and stippling erosion of the cornea. That is, the administration of available cysteamine products has been associated with some stinging and some superficial corneal erosion. The inventor currently believes that those observed. effects may actually result from preservatives in the cysteamine, alone or in combination with the cysteamine. Regardless of the actual cause, the use of cysteamine in those conditions has been publicly associated with stinging and superficial corneal erosion, which would likely lead away from the present invention. In spite of these reports, proceeding against conventional wisdom, the inventor has discovered that cysteamine can be used in the treatment of dysfunctional tear syndrome.

Cysteamine (HS—CH₂—CH—₂—NH₂) is a small sulthydryl compound that is able to cross cell membranes easily due to its small size. Cysteamine plays a role in formation of the protein glutathione (GSH) precursor, and is currently FDA approved for use in the treatment of cystinosis, an intra-lysosomal cystine storage disorder. In cystinosis, cysteamine acts by converting cystine to cysteine and cysteine-cysteamine mixed disulfide, which are then both able to leave the lysosome through the cysteine and lysine transporters respectively (Gahl et al., N Engl J Med 2002; 347(2):111-21). Within the cytosol the mixed disulfide can be reduced by its reaction with glutathione and the cysteine released can be used for further GSH synthesis. Treatment with cysteamine has been shown to result in lowering of intracellular cystine levels in circulating leukocytes (Dohil et al., J. Pediatr 148(6):764-9, 2006).

Cysteamine is also discussed in Prescott et al., Lancet 1972; 2(7778):652; Prescott et al., Br Med J 1978; 1(6116) 856-7; Mitchell et al., Clin. Pharmacol. Ther. 1974; 16(4):676-84; Toxicol. Appl Pharmacol. 1979 48(2):221-8; and Qiu et al., World J Gastroenterol. 13:4328-32, 2007. Unfortunately, the sustained concentrations of cysteamine necessary for a systemic therapeutic effect are difficult to maintain due to rapid metabolism and clearance of cysteamine from the body, with nearly all administered cysteamine, converted to taurine in a matter of hours. These difficulties are transferred to patients in the form of high dosing levels and frequencies, with all of the consequent unpleasant side effects associated with cysteamine (e.g., gastrointestinal distress and body odor). See the package insert for CYSTAGON® (cysteamine, bitartrate). International Publication No. WO 2007/079670 discloses enterically coated cysteamine products and a method of reducing dosing frequency of cysteamine.

Cysteamine is addressed in International. Patent Application Nos. WO 2009/070781, and WO 2007/089670, and U.S. Patent Publication Nos. 20110070272, 20090048154, and 20050245433.

### Dysfunctional Tear Syndrome

The invention is contemplated to be useful with respect to any dysfunctional tear syndrome (DTS), i.e., ocular lubrication dysfunction, whether or not mediated by matrix metalloproteinases, and with or without erosion of surface tissue. Dysfunctional tear syndrome is a complex disease with various components of aqueous deficiency from the lacrimal and accessory lacrimal glands, meibomian gland deficiency or abnormality, and abnormal lipids stimulating evaporation and inflammation of the eyelids. It occurs, from many sources, and can be part of a function of aging and atrophy of the accessory and lacrimal glands. Immunologically mediated conditions can lead to dysfunction and the condition can be exacerbated by a variety of common medications. Most patients present with symptoms of loss or decreased lubrication related to an imbalance in components of tears or a loss of volume of these components. They will then interpret and express these symptoms as itching, pain, dryness, and matted eyelids. The precise diagnosis of DTS has been historically and clinically problematic. Many tests correlate poorly with the patient's symptoms observable findings which often is discomfort and pain. As used herein, in some embodiments, DTS encompasses recurrent corneal erosion and keratitis sicca.

### Matrix Metalloproteinases (MMPs)

In some embodiments, the cysteamine product inhibits enzymatic; activity of one or inure matrix metalloproteinases (MMPs) selected from the group consisting of MMP-1, MMP-2, MMP-3, MMP-4, MMP-5, MMP-6, MMP-7, MMP-8, MMP-9, MMP-10, MMP-11, MMP-12, MMP-13 and MMP-14. In some embodiments, the cysteamine product inhibits the enzymatic activity of MMP-2, MMP-9, MMP-12 and/or MMP-14. MMPs are a group of zinc-dependent endopeptidases implicated in tissue breakdown, including in the eye.

### Cysteamine/Cystamine

Cysteamine plays a role in formation of the protein glutathione (GSH) precursor. In cystinosis, cysteamine acts by converting cystine to cysteine and cysteine-cysteamine mixed disulfide which are then both able to leave the lysosome through the cysteine and lysine transporters respectively (Gahl et al., N Engl J Med 2002; 347(2):111-21). Within the cytosol the mixed disulfide can be reduced by its reaction with glutathione, and the cysteine released can be used for further GSH synthesis. The synthesis of GSH from cysteine is catalyzed by two enzymes, gamma-glutamylcysteine synthetase and GSH synthetase. This pathway occurs in almost types, with the liver being the major producer and exporter of GSH. The reduced cysteine-cysteamine mixed disulfide will also release cysteamine, which, in theory is then able to re-enter the lysosome, bind more cystine and repeat the process (Dohil et al., J Pediatr 2006; 148(6):764-9). In a recent study in children with cystinosis, enteral administration of cysteamine, resulted in increased plasma cysteamine levels, which subsequently caused prolonged efficacy in the lowering of leukocyte cystine levels (Dohil et al.). This may have been due to “re-cycling” of cysteamine when adequate amounts of drug reached the lysosome. If cysteamine acts in this fashion, then GSH production may also be significantly enhanced.

### Cysteamine Products

In another aspect, the disclosure provides cysteamine products for use in the methods described herein.

A “cysteamine product” in the present disclosure refers generally to cysteamine, cystamine, or a biologically active metabolite or derivative thereof, or combination of cysteamine and cystamine, and includes cysteamine or cystamine salts, esters, amides alkylate compounds, prodrugs, analogs, phosphorylated compounds, sulfated compounds, nitrosylated and glycosylated compounds or other chemically modified forms thereof (e.g., chemically modified forms prepared by labeling with radionucleotides or enzymes and chemically modified forms prepared by attachment of polymers such as polyethylene glycol). Thus, the cysteamine or cystamine can be administered in the form of a pharmacologically acceptable salt, ester, amide, prodrug or analog or as a combination thereof. In various embodiments, the cysteamine product includes cysteamine, cystamine or derivatives thereof. In any of the embodiments described herein, a cysteamine product may optionally exclude N-acetylcysteine. The inventor also contemplates compositions that are free of preservatives.

This disclosure contemplates numerous compositions including many possible components. To be clear, when components are described as being contemplated for inclusion, those components are of course also contemplated for exclusion. Unless it is the active ingredient itself, the inventor contemplates excluding any one or more components that are also contemplated for inclusion.

Salts, esters, amides, prodrugs and analogs of the active agents may be prepared using standard procedures known to those skilled in the art of synthetic organic chemistry and described, for example, by J. March, Advanced Organic Chemistry: Reactions, Mechanisms and Structure, 4th Ed. (New York: Wiley-Interscience, 1992). For example, basic addition salts are prepared from the neutral drug using conventional means, involving reaction of one or more of the active agent's free hydroxyl groups with a suitable base. Generally, the neutral form of the drug is dissolved in a polar organic solvent such as methanol or ethanol and the base is added thereto. The resulting salt either precipitates or may be brought out of solution by addition of a less polar solvent. Suitable bases for forming basic addition salts include, but are not limited to, inorganic bases such as sodium hydroxide, potassium hydroxide, ammonium hydroxide, calcium hydroxide, trimethylamine, or the like. Preparation of esters involves functionalization of hydroxyl groups which may be present within the molecular structure of the drug. The esters are typically acyl-substituted derivatives of free alcohol groups, i.e., moieties which are derived from carboxylic acids of the formula R—COOH where R is alkyl, and typically is lower alkyl. Esters can be reconverted to the free acids, if desired, by using conventional hydrogenolysis or hydrolysis procedures. Preparation of amides and prodrugs can be carried out in an analogous manner. Other derivatives and analogs of the active agents may be prepared using standard techniques known to those skilled in the art of synthetic organic chemistry, or may be deduced by reference to the pertinent literature.

In various embodiments, the cysteamine product does not refer to nanoparticles (including, but not limited to, gold, silver, cadmium and iron nanoparticles) comprising cysteamine (e.g., Wu et al, Nanomedicine: Nanotechnology, Biology and Medicine, 8:860,869, 2011: Ghosh et al., Biomaterials, 34:807-816, 2013; Unak et al Surf. N. Niointerfaces, 90:217-226, 2012; Petkova et al., Nanoscale Res. Lett., 7:287, 2012 and U.S. Patent Publication No. 2010/0034735) or cysteamine incorporated into another active agent (e.g., Fridkin et al., J. Comb. Chem., 7:977-986, 2005).

### Pharmaceutical Formulations

The disclosure provides cysteamine products useful in the treatment of dysfunctional tear syndrome (DTS). To administer cysteamine products to patients or test animals, it is preferable to formulate the cysteamine products in a composition comprising one or more pharmaceutically acceptable carriers. Pharmaceutically or pharmacologically acceptable earners or vehicles refer to molecular entities and compositions that do not produce allergic, or other adverse reactions when administered using routes well-known in the art, as described below, or are approved by the U.S. Food and Drug Administration or a counterpart foreign regulatory authority as an acceptable additive to orally or parenterally administered pharmaceuticals. Pharmaceutically acceptable carriers include any and all clinically useful solvents, dispersion media, coatings, antibacterial and antifungal agents, isotonic and absorption delaying agents and the like.

Pharmaceutical carriers include pharmaceutically acceptable salts, particularly where a basic or acidic group is present in a compound. For example, when an acidic substituent, such as —COOH, is present, the ammonium sodium, potassium, calcium and the like salts, are contemplated for administration. Additionally, where an acid group is present, pharmaceutically acceptable esters of the compound (e.g., methyl, tert-butyl, pivaloyloxymethyl, succinyl, and the like) are contemplated as preferred forms of the compounds, such esters being known in the art for modifying solubility and/or hydrolysis. characteristics for use as sustained release or prodrug formulations.

When a basic group (such as amino or a basic heteroaryl radical, such as pyridyl) is present, then an acidic salt, such as hydrochloride, hydrobromide, acetate, maleate, pamoate, phosphate, methanesulfonate, p-toluenesulfonate, and the like, is contemplated as a form for administration.

In addition, compounds may form solvates with water or common organic solvents. Such solvates are contemplated as well.

The cysteamine products are generally administered to the eye, more particularly to the surface, or epithelial tissue of the eye, and more particularly, the cornea. Generally, compositions for administration by any of the above methods are essentially free of pyrogens, as well as other impurities that could be harmful to the recipient. Further, compositions for administration are sterile.

Pharmaceutical compositions of the disclosure containing a cysteamine product as an active ingredient may contain pharmaceutically acceptable carriers or additives depending on the route of administration. Examples of such carriers or additives include water, a pharmaceutically acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxyl, methyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (FBA), mannitol, sorbitol, lactose, a pharmaceutically acceptable surfactant and the like. Additives used are chosen from, but not limited to, the above or combinations thereof, as appropriate, depending on the dosage form of the disclosure.

Formulation of the pharmaceutical composition will vary according to the route of administration selected (e.g., solution, emulsion). An appropriate composition comprising the cysteamine product to be administered can be prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's, or fixed oils. Intravenous vehicles can include various additives, preservatives, or fluid, nutrient or electrolyte replenishers.

A variety of aqueous carriers, e.g., water, buffered water, 0.4% saline, 0.3% glycerine, or aqueous suspensions may contain the active compound in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphande, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene, oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate.

In some embodiments, the cysteamine product disclosed herein can be lyophilized for storage and reconstituted in a suitable carrier prior to use. Any suitable lyophilization and reconstitution techniques can be employed. It is appreciated by those skilled in the art that lyophilization and reconstitution can lead to varying degrees of activity loss and that use levels may have to be adjusted to compensate.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example coloring agents, may also be present.

The inventor also contemplates that the cysteamine may be formulated along with another drug or active agent normally used in the treatment of dry eye, such as artificial tears or an MMP inhibitor.

The concentration of cysteamine product in the inventive formulations can vary widely, for example from less than about 0.01%, usually at or at least about 0.1% to as much as 5 or 10% by weight and are selected primarily based on fluid volumes, manufacturing characteristics, viscosities, etc., in accordance with the particular mode of administration selected. Actual methods for preparing administrable compositions are known or apparent to those skilled in the art and are described in more detail in, for example, Remington's Pharmaceutical Science. Of course, the cysteamine should be formulated in an amount sufficient for treatment of the disease or symptom thereof, and ideally in an amount that does not itself result in irritation of the eye or local tissues. Skilled persons will be readily able to determine amounts within the therapeutic window.

Compositions useful for administration may be formulated with uptake or absorption enhancers to increase their efficacy. Such enhancers include, for example, salicylate, glycocholate/linoleate, glycholate, aprotinin, bacitracin, SDS, caprate and the like See, e.g., (J. Pharm. Sci., 85:1282-1285, 1996) and Oliyai and Stella (Ann. Rev. Pharmacol. Toxicol., 32:521-544, 1993).

### Dosing and Administration

The cysteamine product is administered in a therapeutically effective amount; typically; the composition is in unit dosage form. The amount of cysteamine product administered is, of course, dependent on the age, weight, and general condition of the patient, the severity of the condition being treated, and the judgment of the prescribing physician. Suitable therapeutic amounts can be determined by a skilled person. The dose can be administered once per day, or multiple times per day. The cysteamine product may be administered less than four time per day, e.g., one, two or three times per day.

Administration may continue for at least 3 months, 6 months, 9 months, 1 year, 2 years, or more. The administration may be continued as symptoms continue, or may be continued until symptoms cease. Administration may be continued in a patient with a history of dysfunctional tear syndrome (DTS) as a prophylactic measure.

Dosages may include any amount of cysteamine that is therapeutically effective for treatment of the symptoms. Concentrations of cysteamine in the dosage form will generally range from about 0.001 mg/ml to about 5 mg/ml, or from about 0.01 to about 2 mg/ml, or from about 0.1 to about 1 mg/ml. Concentrations of cysteamine may range from about 0.001, 0.002, 0.004, 0.006, 0.008, 0.01, 0.015, 0.2, 0.3, 0.4, 0.5, 0.6, 0.7, 0.8, or 0.9, to about 0.95, 1.0, 1.1, 1.2, 1.3, 1.4, 1.5, 1.6, 1.7, 1.8, 1.9, 2.0, 2.5, 3.0, 4.0, 5.0, or 10.0 mg/ml. The inventor contemplates ranges of doses from any of the lowest disclosed values to any of the highest disclosed values, such as from about 0.004 mg/ml to about 2.5 or from about 0.015 mg/ml to about 1.2 mg/ml, or any combination of values set forth in this application.

Cysteamine compositions may be provided in multiple dose vials, generally maintained in a refrigerated state after opening, and generally with a limited shelf-life. Alternatively, doses may be single-use vials maintained in a preservative-free state prior to opening. In some embodiments, the compositions are maintained at room temperature before opening (particularly, for example, sterile single-dose vials), or frozen (e.g., in multiple-use vials). The inventor also contemplates lyophilized or otherwise reconstitutable compositions that can be reconstituted by a physician or other practitioner before administration.

### Combination Therapy

Therapeutic compositions can be administered in therapeutically effective dosages alone or in combination with other drugs useful for treatment of dysfunctional tear syndrome (DTS) or its symptoms. Examples of other active agents include cyclosporine, lifitegrast, etc. Other combination agents include artificial tears or other acceptable eye lubricants.

In some embodiments, the methods described herein further comprise administering an additional MMP inhibitor (e.g., an MMP-2 inhibitor, an MMP-12 inhibitor and/or an MMP-9 inhibitor) to the subject. In this regard, the inventor contemplates the addition of another agent that directly or indirectly inhibits MMP activity. This includes an agent that blocks MMP activity or an agent that blocks a pathway of MMP production. The agent causes a reduction in MMP activity in the eye, regardless of the mechanism of its action. Representative examples of MMP inhibitors include Tissue Inhibitors of Metalloproteinases (TIMPs) (e.g., TIMP-1, TIMP-2, TIMP-3, or TIMP-4), O2-macroglobulin, tetracyclines (e.g., tetracycline, minocycline, and doxycycline), hydroxamates (e.g., BATIMASTAT, MARIMISTAT and TROCADE), claelators (e.g., EDTA, cysteine, acetylcysteine, D-penicillamine, and gold salts), synthetic MMP fragments, succinyl mercaptopurines, phosphonamidates, and hydroxaminic acids.

Broad-spectrum inhibitors that inhibit more than one type of MMP are also contemplated. Exemplary broad spectrum MMP inhibitors include, but are not limited to, GM6001, batimastat, marimastat, prinomastat, BAY 12-9566, MMI270(B), BMS-275291, and metastat. Inhibitors that are capable of inhibiting MMP2, MMP9, or both MMP2 and MMP9 are specifically contemplated. An exemplary MMP-2/MMP-9 inhibitor includes, but is not limited to, SB-3CT. For example, in one embodiment, combination therapy comprising the administration of the cysteamine product and a MMP-2/MMP-9 inhibitor is specifically contemplated.

Assays for measuring MMP inhibition/suppression are readily known in the art, and include, for example, the following: Cawston T. E., Barrett A. S., “A rapid and reproducible assay for collagenase using (14C) acetylated collagen,” Anal. Biochem. 35:1961-1965 (1963); Cawston T. E., Murphy G. “Mammalian collagenases,” Methods in Enzymology 80:711 (1981); Koshy P. T. J., Rowan A. D., Life P. F., Cawston T. E., “96-well plate assays for measuring collagenase activity using (3)H-acetylated collagen,” Anal. Biochem. 99:340-345 (1979), Stack M S., Gray R D., “Comparison of vertebrate collagenase and gelatinase using a new fluorogenic substrate peptide,” J. Biol. Chem. 264:4277-4281 (1989); and Knight C. G, Willenbrock F., Murphy G, “A novel coumarin-labelled peptide for sensitive continuous assays of the matrix metalloproteinases,” FEBS Lett 296:263-266 (1991).

The cysteamine product and other drugs/therapies can be administered in combination either simultaneously in a single composition or in separate compositions. Alternatively, the administration is sequential Simultaneous administration is achieved by administering a single composition or pharmacological formulation that includes both the cysteamine product and other therapeutic agent(s). Alternatively, the other therapeutic agent(s) are taken separately at about the same time as a pharmacological formulation tablet, injection or drink) of the cysteamine product.

In various alternatives, administration of the cysteamine product can precede or follow administration of the other therapeutic agent(s) by intervals ranging from minutes to hours. For example, in various embodiments, it is further contemplated that the agents are administered in a separate formulation and administered concurrently, with concurrently referring to agents given within 30 minutes of each other.

In embodiments where the other therapeutic agent(s) and the cysteamine product axe administered separately, one would generally ensure that the cysteamine product and the other therapeutic agent(s) are administered within an appropriate time of one another so that both the cysteamine product and the other therapeutic agent(s) can exert, synergistically or additively, a beneficial effect on the patient. For example, in various embodiments the cysteamine product is administered within about 0.5-6 hours (before or after) of the other therapeutic agent(s). In various embodiments, the cysteamine product is administered within about 1 hour (before or after) of the other therapeutic agent(s).

In another aspect, the second agent is administered prior to administration of the cysteamine composition. Prior administration refers to administration of the second agent within the range of one week prior to treatment with cysteamine, up to 30 minutes before administration of cysteamine. It is further contemplated that the second agent is administered subsequent to administration of the cysteamine composition. Subsequent administration is meant to describe administration from 30 minutes after cysteamine treatment up to one week after cysteamine administration.

### Kits

The disclosure also provides kits for carrying out the methods of the disclosure. In various embodiments, the kit contains, e.g., bottles, vials, ampoules, tubes, cartridges and/or syringes that comprise a liquid (e.g., sterile injectable) formulation or a solid (e.g., lyophilized) formulation. The kits can also contain pharmaceutically acceptable vehicles or carders (e.g., solvents, solutions and/or buffers) for reconstituting a solid (e.g., lyophilized) formulation into a solution or suspension for administration (e.g., by injection), including without limitation reconstituting a lyophilized formulation in a syringe for injection or for diluting concentrate to a lower concentration. Furthermore, extemporaneous injection solutions and suspensions can be prepared from, e.g., sterile powder, granules, or tablets comprising a cysteamine product-containing composition. The kits can also include dispensing devices, such as aerosol or injection dispensing devices, pen injectors, autoinjectors, needleless injectors, syringes, bottles, tubes, and/or needles. In various embodiments, the kit also provides an ocular dosage form, e.g., a liquid or other ocularly acceptable formulation described herein, of the cysteamine product for use in the method. The kit also provides instructions for use.

While the disclosure has been described in conjunction with specific embodiments thereof, the foregoing description as well as the examples which follow are intended to illustrate and not limit the scope of the disclosure. Other aspects, advantages and modifications within the scope of the disclosure will be apparent to those skilled in the art.

### EXAMPLES

Cysteamine ovular drops decreases objective and subjective symptoms of Dry Eye Disease (dysfunctional tear syndrome (DTS), dry eye, and keratitis sicca) in MMP positive patients.

Sterile ocular cysteamine drops with a concentration of 0.44% were used in a dosage schedule of one drop per eye, four times per day.

Four patients were self-treated with cysteamine ocular drops as described above for 30-37 days. Patients were requested to fill out a diary sheet daily and to return weekly for follow-up. Three of the four patients filled out the diary that included a 3-point Likert scale of symptoms, number of times drops were used daily, and a space for other comments. Patients were seen at least 2-5 times following the initial visit. Patients were tested for MMP using the INFLAMMADRY test (see Kaufman) and the Schirmer test (in which a filter paper strip is placed in the lower fornix of the eyelid to absorb tears), with and without anesthesia, using Oxford grading (observation of conjunctiva and cornea using BIO-GLO fluorescein strips) at entry, and Schirmer with and without anesthesia on the final visit.

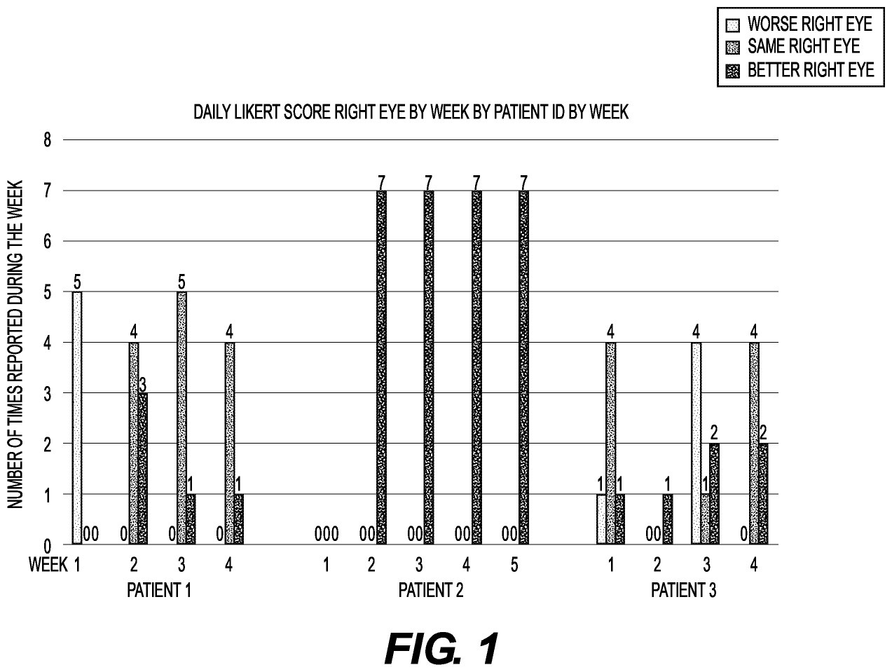

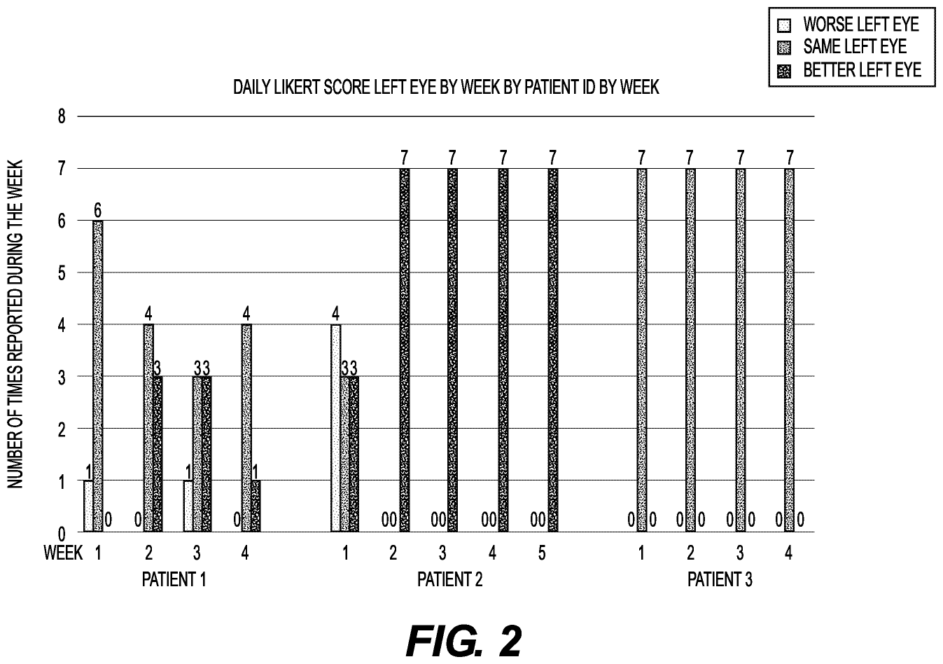

Four patients (Patients 1, 2, 3, and 4) were tested. Patients 1 and 3 tested positive for MMP and had objective measures indicating dysfunctional tear syndrome (DTS); Patient 2 tested negative for MMP and did not have positive objective measures, but had positive subjective symptoms of DTS. Among the patients testing MMP-positive, there was an improvement in Likert-scale and other comments. Additionally, when interviewed during each weekly visit, patients reported changes in symptoms indicative of improvement (FIGS. 1 and 2). These two patients also showed improvement in Schirmer tests at the end of the study. Patients 1 and 3 returned for 4 weekly visits; Patient 2 returned for 5 weekly visits. Patient 2 did not have objective findings of dysfunctional tear syndrome (DTS) and did not report changes in 3-point Likert scale but reported that she was able to go without artificial tears for a majority of the four weeks after starting treatment.

Patient 4 was not tested for MMP, but presented with symptoms of DTS, including years of pain and poor response to other treatments. On the theory the MMP is involved or a causative factor in recurrent epithelial erosion and dry eye, he was treated using the same regimen noted above for Patients 1, 2, and 3. Following treatment, Patient 4 reports being “cured,” and is apparently free of significant symptoms.

These findings confirm both biologic and clinical plausibility that cysteamine optical drops can be used to treat MMP-positive dysfunctional tear syndrome (DTS) symptoms.

All publications and patents mentioned herein are hereby incorporated by reference in their entirety as if each individual publication or patent was specifically and individually indicated to be incorporated by reference. In case of conflict, the present application, including any definitions herein, will control.

1. A method for reducing at least one symptom of dysfunctional tear syndrome (DTS) or ocular lubrication dysfunction comprising administering cysteamine, or a derivative thereof, to a subject exhibiting at least one symptom of DTS or ocular lubrication dysfunction.

2. The method according to claim 1, wherein the subject is a human.

3. The method according to claim 2, wherein the subject is a human diagnosed with recurrent corneal erosion or keratoconjunctivitis sicca.

4. The method according to claim 1, wherein the method comprises administering cysteamine at least once daily.

5. The method according to claim 4, wherein the method comprises administering cysteamine at least twice daily.

6. The method according to claim 1, wherein the administration comprises administering a composition comprising about 0.01 to about 2.0 mg/ml cysteamine.

7. The method according to claim 6, wherein the composition comprises cysteamine in saline.

8. The method according to claim 6, wherein the composition comprises cysteamine in combination with at least one additional active agent.

9. The method according to claim 8, wherein the at least one additional active agent is an MMP-9 inhibitor.

10. The method according to claim 6, wherein the composition comprises cysteamine and at least one pharmaceutically acceptable excipient for alleviating the symptoms of dysfunctional tear syndrome (DTS).

11. The method according to claim 10, wherein the excipient is glycerol.
